(19) United States
(12) Patent Application Publication
(10) Pub. No.: US 2026/0306073 A1
(43) Pub. Date: Oct. 8, 2026

(54) FLUID COLLECTION AND SAMPLING DEVICE

(71) Applicant: BIOXHALE LIMITED, Leicester Leicestershire (GB)

(72) Inventor: Charles Lawrence Paul THOMAS, Loughborough Leicestershire (GB)

(21) Appl. No.: 19/482,289

(22) PCT Filed: May 9, 2024
(86) PCT No.: PCT/GB2024/051201

§ 371 Date: Nov. 7, 2025

(30) Foreign Application Priority Data
May 9, 2023 (GB) 2306828.1

(51) Int. Cl. A61B 10/00; G01N 33/497

(52) U.S. Cl. CPC A61B 10/00; G01N 33/497; A61B 2010/0087

(57) ABSTRACT

A device for collecting a fluid and storing a sample of or from the fluid, the device comprising a first sample chamber volume having a first flow path for receiving the fluid, first selective closure means for selectively closing the first flow path so that fluid may not flow into or out of the first sample chamber volume via the first flow path, a second sample chamber volume in selective fluid communication with the first sample chamber volume, second selective closure means for selectively fluidly isolating the second sample chamber volume from the first sample chamber volume, and fluid moving means for moving fluid from the first sample chamber volume to the second sample chamber volume when the second selective closure means permits fluid flow therebetween. The device is moveable between (i) a first configuration in which fluid may flow into in the first sample chamber volume via the first fluid path and be collected therein whilst the second selective closure means fluidly isolates the second sample chamber volume from the first sample chamber volume, and (ii) a second configuration in which the first fluid path is closed by the first selective closure means, the second selective closure means permits fluid flow between the first sample chamber volume and the second sample chamber volume, and the fluid moving means causes the collected fluid to move into the second sample chamber volume so that a sample of or from the fluid may be stored in the second sample chamber volume.

### TECHNICAL FIELD

The present invention relates to the sampling of a fluid from a person or an animal. Certain embodiments of the invention relate to breath sampling, and particularly but not exclusively to an apparatus and method for expiratory reserve volume sampling.

### BACKGROUND

A typical human lung contains between 6 to 8 litres of air. Normal, “unconscious” breathing is termed tidal breathing and approximately 0.5 litres of air is inhaled and exhaled during each tidal breath cycle. This is known as the tidal volume.

The maximum volume of air that may be inhaled and exhaled is known as the vital capacity, with a volume between 4 to 6 litres, depending on the individual. When we expel all our breath, and empty our lungs, there remains within our lungs a volume of air of between 2 to 2.5 litres; known as the residual volume. The difference between the maximum volume of air we can exhale, and our tidal breathing exhalation is known as the expiratory reserve volume.

These air volumes are illustrated in FIG. 1, which shows typical spirometric measures of lung function.

Capnography (CO₂ monitoring of respiratory gases) classifies a single exhalation of breath into three phases:
Phase I (also known as the dead space) contains inspired air with no gases from the alveolar structures of the lung (alveolar air);
Phase II contains a mixture of inspired air and gases from the alveolar region of the lung; and,
Phase III contains alveolar air.

These three phases are illustrated schematically in FIG. 2.

When we “empty our lungs” and breathe out as much, and for as long as we can, we are extending the phase III of our breath. During this time the concentrations of CO₂, and other exhaled compounds, reach a plateaux value as we exhale our expiratory reserve volume.

It can be desirable to obtain a sample from an individual's expired breath. As well as respiratory gases, such samples also contain volatile organic compounds (VOC), which can provide information about the individual and can be used to diagnose certain medical conditions. Phase Ill alveolar air comprised within the expiratory reserve volume typically contains a relatively high concentration of VOC in comparison with phase I or phase II air. It can thus be desirable to obtain a sample of an individual's expiratory reserve volume. Sampling the expiratory reserve volume from an individual further provides a physiological endpoint for the sample that is reproducible between individuals, as well as ensuring that the concentrations of exhaled circulating compounds sampled are at their highest.

Known methods of sampling the expiratory reserve volume are not standardised, and are typically difficult to use in a manner that is repeatable across individuals. Certain embodiments of the present invention aim to provide a breath sampling method and apparatus that is simple to use, repeatable and clinically safe for both everyone involved in giving and taking the sample.

The same principles may also be applied to the collection of other fluid samples from a person or animal.

### SUMMARY OF THE INVENTION

In accordance with an aspect of the present invention, there is provided a device (e.g. handheld device) for collecting a fluid and storing a sample of or from the fluid, the device comprising:
a first sample chamber volume having a first flow path for receiving the fluid;
first selective closure means for selectively closing the first flow path so that fluid may not flow into or out of the first sample chamber volume via the first flow path;
a second sample chamber volume in selective fluid communication with the first sample chamber volume;
second selective closure means for selectively fluidly isolating the second sample chamber volume from the first sample chamber volume; and
fluid moving means for moving fluid from the first sample chamber volume to the second sample chamber volume when the second selective closure means permits fluid flow therebetween;
wherein the device is moveable between:
a first configuration in which fluid may flow into in the first sample chamber volume via the first fluid path and be collected therein whilst the second selective closure means fluidly isolates the second sample chamber volume from the first sample chamber volume; and
a second configuration in which the first fluid path is closed by the first selective closure means, the second selective closure means permits fluid flow between the first sample chamber volume and the second sample chamber volume, and the fluid moving means causes the collected fluid to move into the second sample chamber volume so that a sample of or from the fluid may be stored in the second sample chamber volume.

The device may be moveable between a pre-use configuration and the first configuration and the second configuration, wherein in the pre-use configuration, the first fluid path is closed by the first selective closure means and fluid flow between the first sample chamber volume and the second sample chamber volume is prevented by the second selective closure means.

The first selective closure means may be manually moveable between a closed position in which the first fluid path is closed and an open position in which the first fluid path is open.

The second selective closure means may be manually moveable between a closed position in which the second sample chamber volume is fluidly isolated from the first sample chamber volume, and an open position in which the second sample chamber volume is in fluid communication with the first sample chamber volume.

That is, the device may be manually moveable between the first configuration and the second configuration.

The device may comprise an inlet collar wherein at least part of the first selective closure means and/or the second selective closure means form part of the inlet collar. The inlet collar may be rotatably moveable so as to move the first selective closure means between the closed position and the open. The inlet collar may be axially moveable so as to move the second selective closure means between the closed position and the open position. The inlet collar may comprise a mouthpiece and the device is a device for collecting breath and storing a sample of or from the breath.

The mouthpiece may have a mouthpiece inlet and at least one mouthpiece outlet, and the first sample chamber volume has at least one chamber aperture;
wherein the mouthpiece is moveable (e.g. manually moveable) between:
   a first position in which the at least one mouthpiece outlet is occluded, thereby closing the first flow path;
   a second position in which the at least one mouthpiece outlet is at least partly overlapping with the at least one chamber aperture, permitting air to flow through the first flow path from the mouthpiece inlet through the at least one chamber aperture into the first sample chamber volume; and
   a third position in which the at least one mouthpiece outlet is not overlapping with the at least one chamber aperture, permitting air to flow through a second flow path from the first sample chamber volume through the at least one chamber aperture into the second sample chamber volume.

The third position may lie beyond the second position, such that the mouthpiece must move through the second position to reach the third position.

The mouthpiece may be rotatable in a first direction between the first position and the second position.

The mouthpiece may be displaceable in a second direction between the second position and the third position, the second direction being different to the first direction.

The mouthpiece may further comprise a sealing member operable to seal against an inlet of a sample medium.

Air flow into a sample medium held in the receiving aperture may be prevented when the mouthpiece is in the first position or in the second position.

Air flow from the mouthpiece inlet into the first sample chamber volume may be prevented when the mouthpiece is in the first position or in the third position.

The chamber aperture may be adjacent to the receiving aperture.

The device may further comprise a first valve fluidly connected downstream of a valve outlet of the second sample chamber volume, the first valve having an open position in which air is permitted to flow through the first valve and a closed position in which air flow through the first valve is prevented.

The first valve may comprise a pinch valve, said pinch valve optionally comprising an actuator and a tube compressible by the actuator. The first valve may be provided in a valve chamber connectable to the sample chamber.

The fluid moving means may comprise a vacuum source operable, when fluidly connected to the second sample chamber volume with the mouthpiece in the third position, to draw air within the first sample chamber volume through a sample medium held in the receiving aperture.

The fluid moving means may comprise a vacuum source, such that when the vacuum source is in fluid communication with the second sample chamber volume a pressure difference causes the collected fluid to move into the second sample chamber volume. The vacuum source may comprise a vacuum chamber containing a vacuum or a gas having lower than atmospheric pressure. The device may comprise a vacuum valve for selectively fluidly connecting the vacuum chamber to the second sample chamber volume.

The vacuum chamber may be moveable relative to the sample chamber and movement of the vacuum chamber relative to the sample chamber may cause the vacuum valve to open and fluidly connect the vacuum chamber to the second sample chamber volume.

The vacuum chamber may be rotatably and/or axially moveable relative to the sample chamber and rotational and/or axial movement of the vacuum chamber relative to the sample chamber may cause the vacuum valve to open and fluidly connect the vacuum chamber to the second sample chamber volume.

The second sample chamber volume may comprise a sample medium.

The first sample chamber volume may further comprise one or more exhaust outlets, said exhaust outlet optionally or preferably comprising a filter.

The first sample chamber volume may comprise a tortuous airflow path having an upstream end adjacent the first flow path and a downstream end adjacent the at least one exhaust outlet.

The sample chamber may comprise a plurality of nested concentric walls or baffles defining the tortuous airflow path.

According to another aspect of the invention we provide a breath sample collection apparatus comprising:
a mouthpiece having a mouthpiece inlet and at least one mouthpiece outlet;
a sample chamber in fluid communication with the mouthpiece, the sample chamber having a receiving area operable to receive a sample medium and at least one chamber inlet region, such as a chamber aperture.

The mouthpiece is moveable between:
a first position in which the at least one mouthpiece outlet is occluded, preventing airflow into the sample chamber;
a second position in which the at least one mouthpiece outlet is at least partly overlapping with the at least one chamber inlet region, permitting air to flow through a first flow path from the mouthpiece inlet through the at least one chamber inlet region into the sample chamber; and
a third position in which the at least one mouthpiece outlet is not overlapping with the at least one chamber inlet region, permitting air to flow through a second flow path from the sample chamber through the at least one chamber inlet region into a sample medium held in the receiving aperture.

A breath sampling apparatus of the type described herein may be used to receive a breath sample from an individual and to seal the received sample within the sample chamber for transfer to a testing facility.

The third position may lie beyond the second position, such that the mouthpiece must move through the second position to reach the third position. This may help to prevent accidental contamination of a sample medium held in the receiving area until after a sample has been taken.

The mouthpiece may be moveable (e.g. rotatable) in a first direction between the first position and the second position. The mouthpiece may be moveable (e.g. displaceable) in a second direction between the second position and the third position, the second direction being different to the first direction. Providing different movement directions between the first and second position and second and third positions may help to prevent accidental movement directly to the third position from the first position.

The mouthpiece may further comprise a sealing member operable to seal against an inlet of a sample medium. The mouthpiece may further comprise an end stop operable to prevent air being exhaled directly into the sample tube when a user exhales into the inlet of the mouthpiece. An underside of the end stop may comprise the sealing member. These features may assist in preventing air ingress into the sample medium until after a sample has been collected.

Air flow into a sample medium held in the receiving aperture is preferably prevented when the mouthpiece is in the first position or in the second position, or when moving between the first position and the second position. This helps to ensure isolation of the sample medium during sample collection.

Air flow from the mouthpiece inlet into the sample chamber is preferably prevented when the mouthpiece is in the first position or in the third position. This helps to prevent contamination of the sample medium by external air.

The chamber inlet region (e.g. the at least one chamber aperture) may be adjacent the receiving aperture. This helps to ensure that the volume of exhaled air which enters the sample chamber last is the volume of air which enters the sample medium first.

The sample chamber may further comprise one or more exhaust outlets, said exhaust outlet optionally or preferably comprising a filter. An outlet improves ease of use for a user of the breath sampling apparatus by reducing pressure build up within the device during exhalation. A filter may prevent potentially harmful components of the exhaled breath (e.g. pathogens) from escaping the sample chamber.

The sample chamber may comprise a tortuous airflow path having an upstream end adjacent the chamber aperture and a downstream end adjacent the at least one exhaust outlet. A plurality of nested concentric walls may define the tortuous airflow path.

The breath sample collection apparatus may further comprise a first valve having an open position in which air is permitted to flow through the first valve and a closed position in which air flow through the first valve is prevented. The first valve may be fluidly connected to a valve outlet of the sample chamber. The first valve may comprise a pinch valve, said pinch valve optionally comprising an actuator and a tube compressible by the actuator. The first valve may be provided in a valve chamber connectable to the sample chamber.

The breath sample collection apparatus may comprise an adapter operable to couple to the sample medium. The adapter may be removably connected to the valve chamber. Such a removable adaptor may be interchanged, to permit use of the breath sample collection apparatus with a plurality of types or shapes of sample media.

The breath sample collection apparatus may further comprise a non-return valve operable to permit air to flow through the (or a) valve outlet of the sample chamber but to prevent air flow in the reverse direction. The non-return valve may be comprised in one or both of the adapter and the valve chamber. Such a non-return valve may prevent contamination of a sample when the first valve is opened.

The breath sample collection apparatus may further comprise a vacuum source operable, when fluidly connected to the sample chamber with the mouthpiece in the third position, to draw air within the sample chamber through a sample medium held in the receiving aperture.

The vacuum source may comprise a vacuum chamber connectable to the valve chamber, such that the vacuum chamber is operable to draw air within the sample chamber through a sample medium held in the receiving aperture when the first valve is in the open position.

The vacuum chamber may comprise a one way valve through which air within the vacuum chamber may be removed so as to create a vacuum within the vacuum chamber.

The features set out above may be combined with the features of the first aspect of the invention in any combination, and also with features selected from the detailed description below.

### BRIEF DESCRIPTION OF THE DRAWINGS

There now follows a detailed description of the invention, by way of example only, with reference to the accompanying drawings, in which:

FIG. 1 illustrates typical spirometric measures of lung function;

FIG. 2 is an example capnograph showing how concentration of CO in an exhaled breath changes across three phases;

FIG. 3 shows a breath sample collection apparatus in accordance with an embodiment of the present invention;

FIG. 4 shows a cross sectional view of the breath sample collection apparatus of FIG. 3;

FIG. 5 shows an exploded view of the breath sample collection apparatus of FIG. 3;

FIG. 6A shows the breath sample collection apparatus of FIG. 3 in a pre-use configuration;

FIG. 6B shows a cross-sectional view of a part of the breath sample collection apparatus in the same configuration as shown in FIG. 6A;

FIG. 7A shows the breath sample collection apparatus of FIG. 6A with the cap removed in a first configuration;

FIG. 7B shows a cross-sectional view of a part of the breath sample collection apparatus in the same configuration as shown in FIG. 7A;

FIG. 8 shows a user breathing into the breath sample collection apparatus of FIG. 3;

FIG. 9 shows the breath sample collection apparatus of FIG. 7A with the cap being replaced;

FIG. 10A shows the breath sample collection apparatus of FIG. 9 with the mouthpiece rotated;

FIG. 10B shows a cross-sectional view of a part of the breath sample collection apparatus in the same configuration as shown in FIG. 10A;

FIG. 11A shows the breath sample collection apparatus of FIG. 10A in a second configuration with the vacuum chamber rotated relative to the sample chamber and mouthpiece;

FIG. 11B shows a cross-sectional view of a part of the breath sample collection apparatus prior to the configuration shown in FIG. 11A;

FIG. 11C shows a cross-sectional view of a part of the breath sample collection apparatus in the same configuration as shown in FIG. 11A;

FIG. 12A shows the breath sample collection apparatus of FIG. 11A with the mouthpiece rotated relative to the sample chamber and vacuum chamber;

FIG. 12B shows a cross-sectional view of a part of the breath sample collection apparatus in the same configuration as shown in FIG. 12A;

FIG. 13 shows an exemplary breath sample collection apparatus in accordance with another embodiment of the present invention in (A) front view, side view and (C) rear view;

FIG. 14 shows the breath sample collection apparatus of FIG. 13 in exploded view;

FIG. 15 shows a first cross section A-A through the breath sample collection apparatus of FIG. 13;

FIG. 16 shows a second cross section B-B through the breath sample collection apparatus of FIG. 13;

FIG. 17 shows a cross section through a sample chamber including a sample medium and a mouthpiece, depicting the mouthpiece in a plurality of different positions (i)-(v);

FIG. 18 shows the sample chamber of FIG. 16 with mouthpiece removed in (A) front view, (B) side view, (C) cross-sectional view J-J, (D) plan (top) view and (E) bottom view;

FIG. 19 show the mouthpiece of FIG. 16 removed from a sample chamber in (A) front view, (B) side view, (C) cross-sectional view N-N, (D) plan view and (E) bottom view;

FIG. 20 shows a cross-section similar to view (ii) of FIG. 17, with the sample medium removed;

FIG. 21 shows a valve section of the breath sample collection apparatus in (A) front view, (B) side view, (C) plan view; (D) bottom view and (E) front view with sample medium and valve mechanism removed;

FIG. 22 shows (A) a first cross section D-D through the valve section of FIG. 21 and (B) a second cross section E-E through the valve section of FIG. 21;

FIG. 23 shows cross sections similar to FIG. 22 with sample medium and valve mechanism removed;

FIG. 24 shows a vacuum chamber in (A) front view, (B) side view, (C) in cross section F-F and (D) in cross section G-G; and

FIG. 25 illustrates air flow within the sample chamber in (i) an exhalation step and (ii) a collection step.

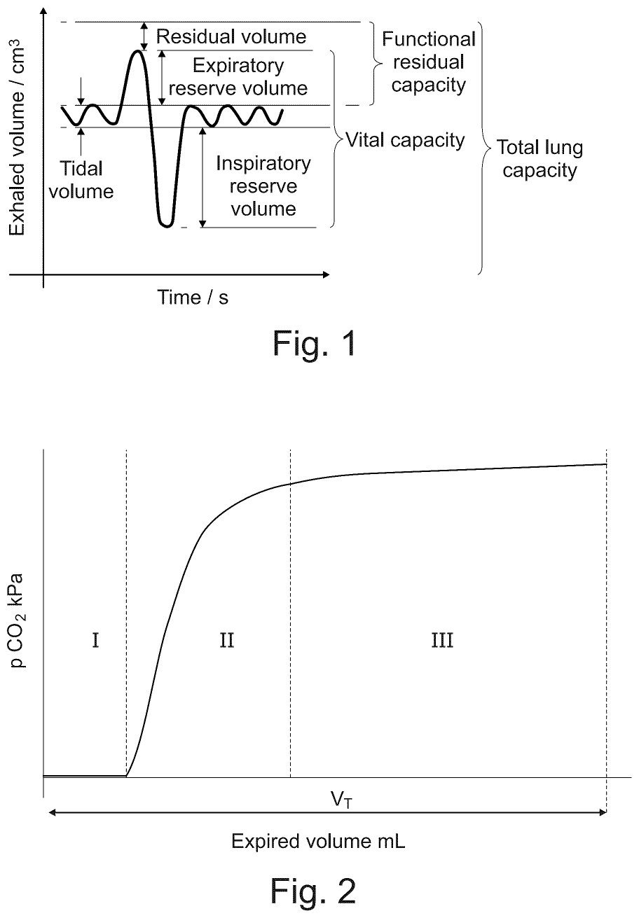

### DETAILED DESCRIPTION

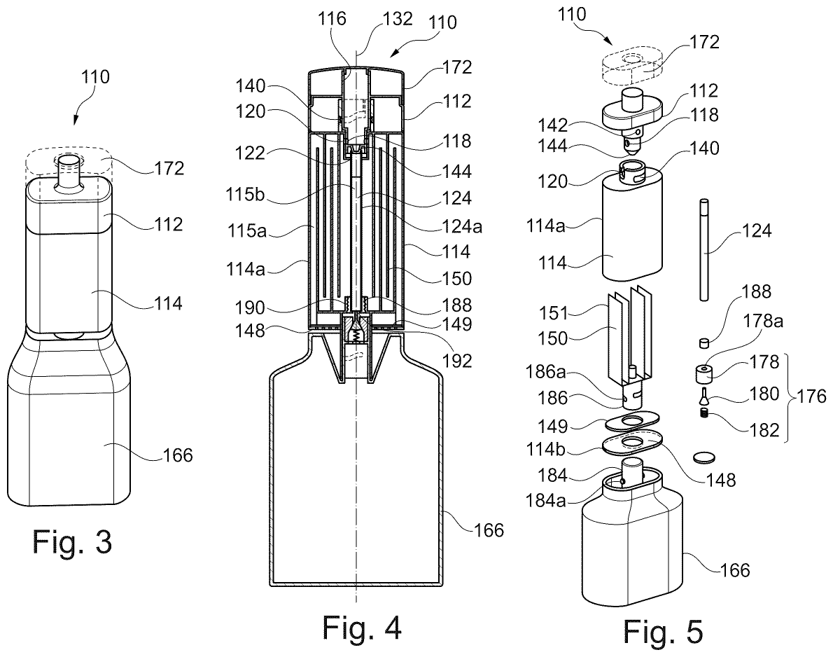

FIG. 3 shows a device 110 for collecting a fluid from a person or animal and storing a sample of or from the fluid in accordance with an embodiment of the present invention. In the non-limiting example shown in FIG. 3, the device 110 is a breath sample collection apparatus. However, in other embodiments, the device 110 may be configured to collect fluids other than breath from a user and store samples of or from such fluids. Such fluids include liquids and gases and may include, as non-limiting examples, urine or blood.

Whilst the following description relates to embodiments that are configured for breath collection and sampling, the described principles and components may be applied to the collection and sampling of other fluids from a person or animal.

The breath sample collection apparatus 110 of FIG. 3 is a handheld device and comprises a sample chamber 114, a valved mouth piece 112 covered by a removable cap 172, and a vacuum chamber 166. As is described in more detail below, a user of the apparatus 110 may remove the cap 172 and blow into the mouthpiece 112 so that a breath sample may be captured in a sample medium and stored within the apparatus 110. The apparatus 110 may then be transported with the captured breath sample sealed therein for analysis of the breath sample at an appropriate testing facility. In certain embodiments, the apparatus 110 requires no electrical power to operate. In particular all movement of components may be actuated manually by the user, and no electrical power may be required to open valves or fluid pathways. That is, the device may be manually operable.

By being a handheld apparatus 110, the apparatus is sized and otherwise configured so that it may be handled and operated by the user with one or two hands.

The handheld breath sample collection apparatus 110 may be entirely self-contained in the sense that all of its features are contained in the handheld device. That is, no wires and/or umbilicals are needed to connect a handheld part of the apparatus 110 to another part of the apparatus (that may render the apparatus, as a whole, as not being handheld).

FIGS. 4 and 5 show a cross-sectional view and an exploded view of the apparatus 110 of FIG. 3, respectively. In FIG. 4, it can be seen that the mouthpiece 112 comprises a mouthpiece inlet 116 and a mouthpiece outlet 118. The mouthpiece 112 is moveable relative to the sample chamber 114. In the non-limiting embodiments shown in the Figures, the mouthpiece 112 is both rotatable relative to the sample chamber 114 about a longitudinal axis 132 and axially moveable relative to the sample chamber 114 in directions parallel to the longitudinal axis.

The sample chamber 114 includes a chamber aperture 120 proximate to the mouthpiece 112. The mouthpiece 112 is moveable relative to the sample chamber 114 so as to selectively align the mouthpiece outlet 118 with the chamber aperture 120. When the mouthpiece outlet 118 is aligned with the chamber aperture 120, a first flow path 128 is opened thereby permitting fluid (i.e. breath) to flow from the mouthpiece 112 into the sample chamber 114. When the mouthpiece outlet 118 is not aligned with the chamber aperture 120, the first flow path 128 is closed such that fluid may not flow into or out of the sample chamber 114 through the chamber aperture 120.

The mouthpiece 112 is coupled to the sample chamber 114 by a pair of diametrically opposed lugs 142 of the mouthpiece 112 disposed in a guide 140 of the sample chamber 114. The guide 140 extends helically so that upon rotation of the mouthpiece 112 relative to the sample chamber 114, the mouthpiece 112 translates axially relative to the sample chamber 114 as well as moving relatively rotationally. In certain embodiments, a screw thread arrangement may achieve a similar effect. Such movement permits the mouthpiece outlet 118 to be moved from a position in which it is aligned with the chamber aperture 120 (and the first flow path 128 is open) and a position in which it is not aligned with the chamber aperture 120 (and the first flow path 128 is closed).

In alternative embodiments, the mouthpiece 112 may be otherwise coupled to the sample chamber 114. For example, a different number of lugs (or an entirely different coupling mechanism) may be employed. In certain embodiments, the lugs may be provided on the sample chamber 114 and the guide 140 may be provided on the mouthpiece.

The mouthpiece outlet 118 and the chamber aperture 120 form first selective closure means for selectively closing the first flow path. In alternative embodiments, the first selective closure means may take on other forms that selectively permits the first flow path to be closed and opened. The first selective closure means may be provided on a component other than the mouthpiece such as an inlet collar that is distinct from the mouthpiece. Such an inlet collar may be coupled with or proximate to the mouthpiece 112 or provided on an apparatus 110 that does not include a mouthpiece 112 at all. The inlet collar may be disposed at an opening of the first fluid path, wherein the at least part of the first selective closure means and/or the second selective closure means form part of the inlet collar. The inlet collar may be rotatably moveable so as to move the first selective closure means between the closed position and the open position. The inlet collar may be axially moveable so as to move the second selective closure means between the closed position and the open position.

Initially, with the first flow path open, the user's breath may be received in a first sample chamber volume 115a within the sample chamber 114. The first sample chamber volume 115a is provided with a plurality of baffles 150 defined by axially extending walls that create a tortuous path for fluid received in the first sample volume 115a. The tortuous path acts as a concertinaed Haldane Tube to segment and isolate the different phases of breath within the sample chamber 114.

In certain embodiments, a diffusional barrier may be incorporated into the tortuous path to impede the ingress of external contaminants into the internal sample transfer volume (i.e. the volume immediately surrounding and including the sample medium). For example, this feature may reduce the ingress of a pure hexane vapour to 1.2×10⁻⁶ g s⁻¹, or at a high environmental contamination level of 1 g m⁻³ to 1×10⁻⁹ g s⁻¹.

In general, the speed of diffusion is limited by the cross-section of the channel along which diffusion takes place. Therefore, in certain embodiments of the invention, some or all fluidic channels within the apparatus 110 are configured to delay diffusion of impurities into the sample chamber 114.

In the embodiment shown in FIGS. 3 to 12, some of the baffles 150 are provided on a baffle insert 151 that is insertable into a main body 114a of the sample chamber 114, whilst other baffles 150 are integrally formed with the interior of the main body 114a. The baffle insert 151 is secured in the main body 114a by a base plate 114b that is connectable to the main body 114a to enclose the first sample chamber volume 115a. A filter 149 (e.g. an FFP3 or N99 filter) is provided adjacent to the base plate 114b to prevent potentially harmful components of the exhaled breath (e.g. pathogens) from escaping the sample chamber 114 through exhaust outlets 148 provided in the base plate 114b. The exhaust outlets 148 permit some of the breath received within the first sample chamber volume 115a to be vented away after travelling along the tortuous path defined by the baffles 150, thereby avoiding pressure build up within the first sample chamber volume 115a.

The sample chamber 114 includes a receiving aperture 122 that may receive and retain a sample medium 124 within the sample chamber 114. In embodiments in which the device is 110 is a breath sample collection apparatus, the sample medium 124 is operable to trap one or more exhaled breath components, such as VOC, in a conventional manner and may for example be a thermal desorption tube, needle-trap, solid-phase microextraction (SPME) trap or silicone substrate. In other embodiments, the sample medium 124 may comprises a C-18 column (e.g. for the recovery of non-volatile compounds from condensed phases, such as urine), PVA embellished surfaces (e.g. for trapping microorganisms from any media), and/or chelating agents (e.g. for trapping heavy metals from condensed phases).

The sample medium 124 is surrounded by a sample medium housing 124a that is tubular with an opening at each end such that the sample medium 124 is only exposed at the ends of the sample medium housing 124a.

Whilst a top end of the sample medium 124 is received in the receiving aperture 122, a bottom end of the sample medium 124 is received in a socket 190 formed within the sample chamber 114. When received in this configuration, the sample medium housing 124a defines a second sample chamber volume 115b that includes the sample medium 124 and that is in selective fluid communication with the first sample chamber volume 115a.

A socket seal 188 is provided in the socket 190 to provide circumferential sealing of the sample medium housing 124a in the socket 190. In a base of the socket, a socket opening 192 is provided which is discussed below. The socket 190 may be configured to receive multiple, different sample media 124, depending on the intended use of the apparatus 110.

The mouthpiece 112 includes a sealing member 144 that may cooperate with the top open end of the sample medium housing 124a to selectively close the second sample chamber volume 115b. As described above, the mouthpiece 112 is axially moveable relative to the sample chamber 114 (and therefore sample medium housing 124a). By moving axially, the sealing member 144 may move between a sealing configuration in which it isolates the second sample chamber volume 115b from the first sample chamber volume 115a and an open configuration in which the second sample chamber volume 115b is in fluid communication with the first sample chamber volume 115a. In this sense, the sealing member 144 serves as a second selective closure means that selectively fluidly isolate the second sample chamber volume 115b from the first sample chamber volume 115a. In other embodiments, any suitable alternative second selective closure means may be provided in place of the axially translating sealing member 144. Such alternative second selective closure means include any means suitable of isolating the sample medium 124 from the first sample chamber volume 115a.

The sample chamber 114 is connected to the vacuum chamber 166 by lugs 184a of the vacuum chamber 166 that are engageable with a guide channel 186a of the baffle insert 151. In particular, the guide channel 186a is disposed on a first stem 186 of the baffle insert 151. When assembled so that the baffles 150 of the baffle insert 151 are disposed within the first sample chamber volume 115, the first stem 186 extends through an aperture of the base plate 114b and is received over a second stem 184 of the vacuum chamber 166 that includes the lugs 184 so that the lugs 184 are received in the guide channel 186a. In alternative embodiments, the first stem 186 may be receivable in the second stem 184 and/or the lugs 184a may be formed on the first stem 186 whilst the guide channel 186a may be disposed on the second stem 184. In certain embodiments, the first stem 184 may be integrally formed with the main body 114a or the base plate 114b of the sample chamber 114.

The guide channel 186a extends helically so that upon rotation of the sample chamber 114 relative to the vacuum chamber 166, the sample chamber 114 translates axially relative to the vacuum chamber 166 as well as moving relatively rotationally.

In certain non-limiting embodiments, the lugs 184a may be provided as a pair of diametrically opposed lugs. In alternative embodiments, the sample chamber 114 may be otherwise coupled to the vacuum chamber 166. For example, a different number of lugs (or an entirely different coupling mechanism, e.g. a screw thread) may be employed.

Disposed within the second stem 184 is a vacuum valve 176. The vacuum valve 176 includes a valve housing 178 having a through bore 178a, a valve seal 180 and a biasing member 182, which in the non-limiting depicted embodiment, is a spring. In alternative embodiments, other biasing members may be employed.

The biasing member 182 provides a biasing force to act on the valve seal 180, causing it to seal it against the valve housing 178 to close the through bore 178a. When the vacuum valve 176 is assembled in the vacuum chamber 166, the valve housing 178 seals against the second stem 184 such that the only permitted fluid pathway from one side of the vacuum valve 176 to the other is via the through bore 178a when it is not closed by the valve seal 180. The valve seal 180 must therefore be acted upon against the biasing force provided by the biasing member 182 in order to open the vacuum valve 176.

In a first configuration of the apparatus 110 as shown in FIG. 4, the vacuum valve 176 is closed such that fluid may not flow across the vacuum valve 176. In this configuration, the valve seal 180 extends into the socket opening 192 adjacent the sample medium 124. Upon axial movement of the sample chamber 114 towards to the vacuum chamber 166 (as the lugs 184a move along guide channels 186a), the sample medium 124 acts against the valve seal 180 to act against the biasing member 182 and open the vacuum valve 176.

The vacuum chamber 166 contains a vacuum (or low pressure gas) such that opening of the vacuum valve 176 causes fluid to flow into the vacuum chamber 166, as is described in more detail below. In alternative embodiments, any suitable fluid moving means may be provided for moving fluid from the first sample chamber volume 115a to the second sample chamber volume 115b when the second selective closure means permits fluid flow therebetween. For example, in place of a vacuum chamber 166, a pump or other vacuum source may be provided.

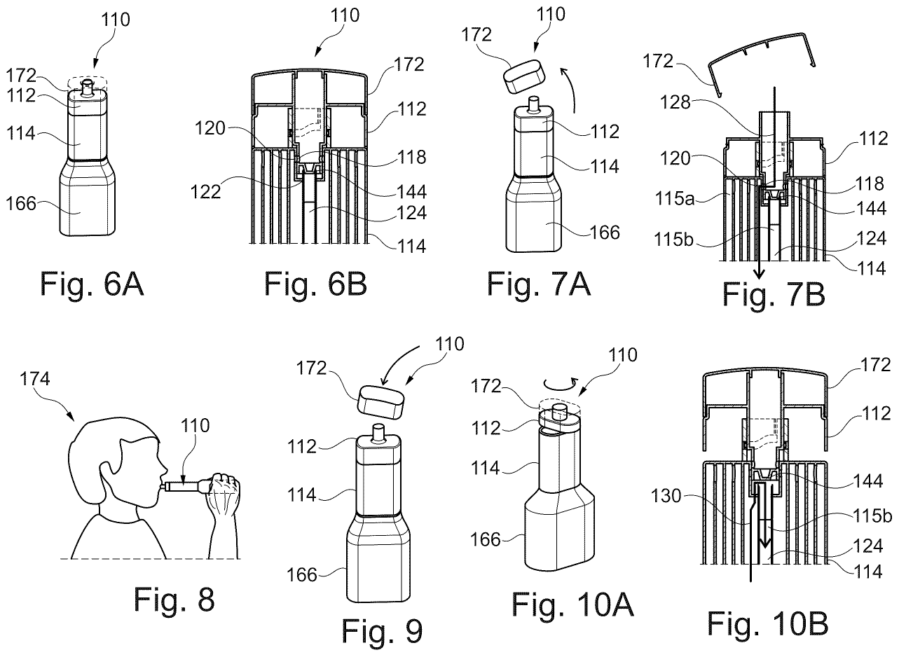

A method of using the apparatus 110 is described below with reference to FIGS. 6A to 12B. FIG. 6A shows the apparatus 110 in a pre-use configuration and FIG. 6B shows a cross-sectional view corresponding to the apparatus 110 and configuration of FIG. 6A. In this configuration, the cap 172 is in place on the mouthpiece 112.

FIG. 7A shows the apparatus 110 in a first configuration, that is suitable for collection of a fluid from a human or animal. FIG. 7B shows a cross-sectional view corresponding to the apparatus 110 and configuration of FIG. 7A. As shown in FIG. 7B, in the first configuration, the mouthpiece outlet 118 is aligned with the chamber aperture 120, and the first flow path 128 is opened thereby permitting fluid (i.e. breath) to flow from the mouthpiece 112 into the first sample chamber volume 115a. In the first configuration, the sealing member 144 fluidly isolates the second sample chamber volume 115b from the first sample chamber volume 115a.

FIG. 8 shows a user 174 blowing into the mouthpiece 112 of the apparatus 110 in the first configuration, thereby passing breath along the first flow path 128 into the first sample chamber volume 115a. Since, in the first configuration, the second sample chamber volume 115b is isolated from the first sample chamber volume 115a, the breath remains in the first sample chamber volume 115a. Excess breath beyond the volume of the first sample chamber volume 115a vents out of the exhaust outlets 148.

Following this step, the cap 172 is replaced on the mouthpiece 112 as indicated in FIG. 9. Subsequently, the mouthpiece 112 (and connected cap 172) are rotated relative to the sample chamber 114 as shown in FIG. 10A. FIG. 10B shows the apparatus 110 of FIG. 10A following a 90 deg rotation of the mouthpiece 112 (and connected cap 172). As shown in FIG. 10B, this relative rotation causes the mouthpiece 112 to move axially away from the sample chamber 114, thereby axially moving the sealing member 144 out of engagement with the sample medium housing 124a and opening a second flow path 130 permitting fluid to flow from the first sample chamber volume 115a to the second sample chamber volume 115b (and hence into the sample medium 124). However, at this stage of operation, the vacuum valve 176 remains closed so opening of the second flow path 130 alone does not necessarily result in movement of the breath (or at least any significant portion of it) from the first sample chamber volume 115a to the sample medium 124 in the second sample chamber volume 115b.

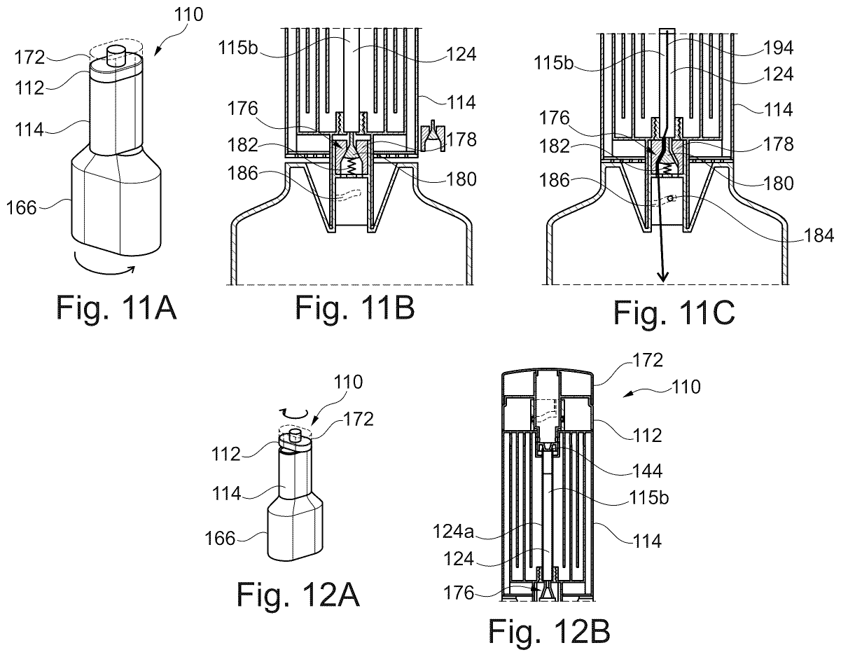

In order to open the vacuum valve 176, the sample chamber 114 is rotated relative to the vacuum chamber 166 as indicated in FIG. 11A. FIG. 11B shows a cross-sectional view of the apparatus 110 of FIG. 11A prior to rotation of the sample chamber 114 relative to the vacuum chamber 166, and FIG. 11C shows a cross-sectional view of the apparatus 110 of FIG. 11A following rotation of the sample chamber 114 relative to the vacuum chamber 166. In the manner described above, as the sample chamber 114 is rotated relative to the vacuum chamber 166, the sample chamber 114 is caused to move axially towards to the vacuum chamber 166 (as the lugs 184a move along guide channels 186a), and the sample medium 124 acts against the valve seal 180 to act against the biasing member 182 and open the vacuum valve 176.

Opening of the vacuum valve 176 puts the second sample chamber volume 115b in fluid communication with the vacuum chamber 166 thereby permitting flow of fluid from the first sample chamber volume 115a towards the vacuum chamber 166 via the sample medium 124 in the second sample chamber volume 115b along a third flow path 194 (shown in FIG. 11C). As the breath contained in the first sample chamber volume 115a travels along the third flow path 194 towards the vacuum chamber 166, one or more exhaled breath components (such as VOCs) are trapped in the sample medium.

Following this step, the sample chamber 114 is rotated relative to the vacuum chamber 116 to once more close the vacuum valve 176 thereby sealing a lower end of the second sample chamber volume 115b. Similarly, the mouthpiece 112 (and connected cap 172) is rotated relative to the sample chamber 114 (as shown in FIG. 12A) to once more close an upper end of the second sample chamber volume 115b by bringing the sealing member 144 into engagement with the sample medium housing 124a. FIG. 12B shows a cross-sectional view of the apparatus 110 of FIG. 12A following rotation of the mouthpiece 112 relative to the sample chamber 114, with the sample contained in the sample medium 124 and sealed in the second sample chamber volume 115b.

When using the apparatus 110 at higher altitudes (where the air pressure is lower than that at lower altitudes), it may be preferable to obtain a larger sample volume. In such cases, the fluid moving means for moving fluid from the first sample chamber volume 115a to the second sample chamber volume 115b (e.g. the vacuum source) may be adjusted to draw the required sample volume.

In certain non-limiting embodiments, the sample volume obtained may be between 100 cm³ and 400 cm³, between 200 cm³ and 300 cm³, or about 250 cm³. For a target sample volume of about 250 cm³, the pressure in the vacuum chamber 166 may be between 0.55 and 0.6 Bar. Pressure and temperature correction may require larger sample volumes with concomitant reductions in the vacuum chamber pressure and increases in maximum flow.

In certain embodiments, the apparatus 110 may be used for field or clinical studies. In certain embodiments, the apparatus 110 may be used to collect environmental samples.

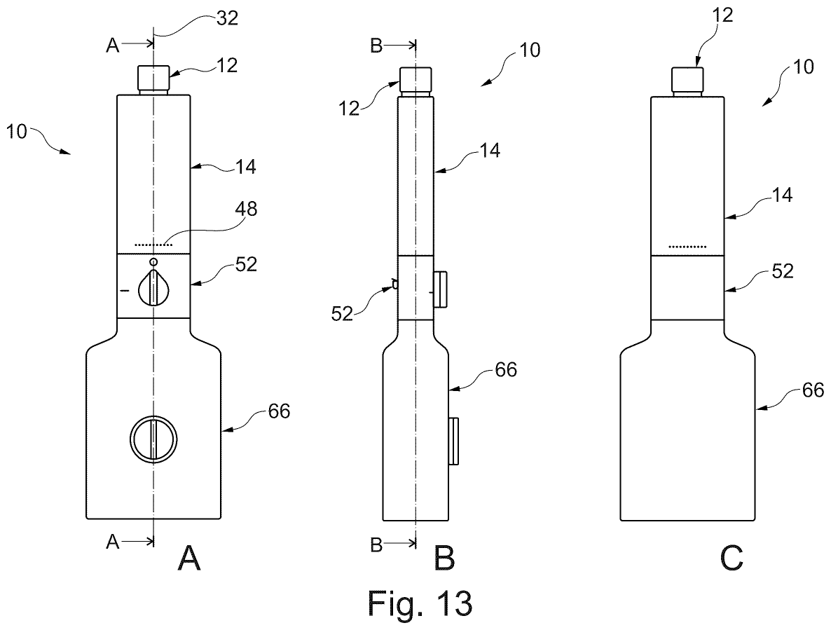

A breath sample collection apparatus 10 (also referred to herein a breath sampling apparatus) according to another embodiment of the present invention is shown in FIG. 13. The breath sample collection apparatus 10 comprises many features that correspond to those described above in relation to the breath sample collection apparatus 110. Like or similar components of the breath sample collection apparatus 10 have the same reference numeral as used above in relation to the breath sample collection apparatus 110, but with the leading ‘1’ removed. The breath sample collection apparatus 10 includes a mouthpiece 12 and a sample chamber 14 in fluid communication with the mouthpiece.

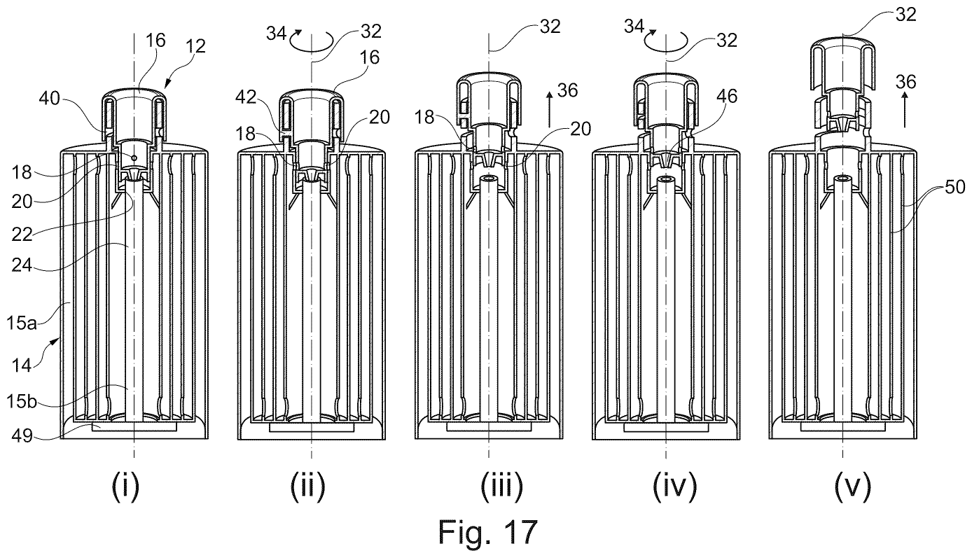

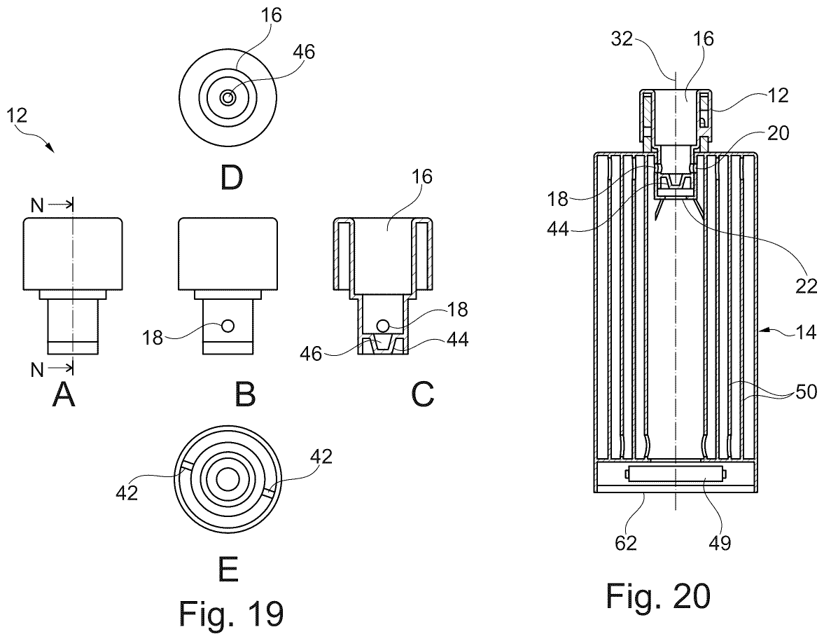

As best shown in FIGS. 17 and 19, the mouthpiece 12 includes a mouthpiece inlet 16 and at least one mouthpiece outlet 18. In the example shown, the mouthpiece includes two spaced apart mouthpiece outlets 18, shown as being diametrically opposed to one another.

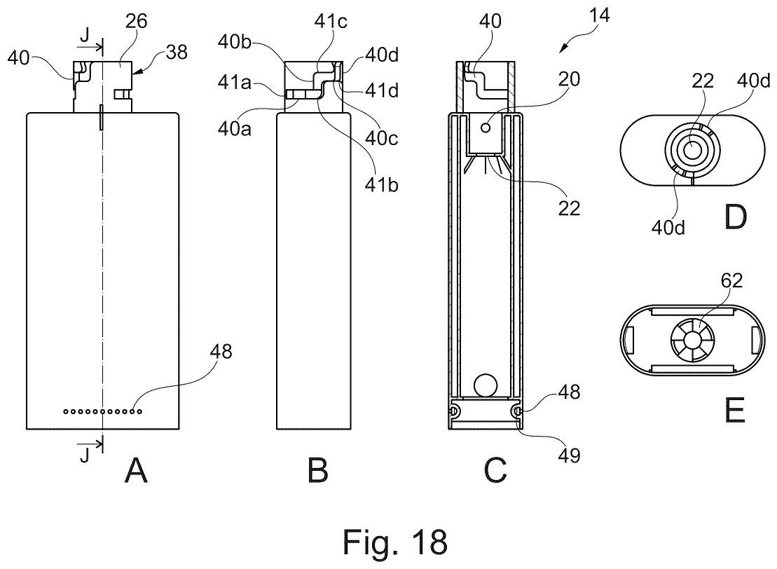

As shown best in FIGS. 17 and 18, the sample chamber 14 includes at least one chamber aperture 20, and in this case two spaced apart chamber apertures (for example, diametrically opposed to one another). The sample chamber further includes a receiving aperture 22 that is operable to receive a sample medium 24. The sample medium 24 is operable to trap one or more exhaled breath components, such as VOC, in a conventional manner and may for example be a thermal desorption tube, needle-trap, solid-phase microextraction (SPME) trap or silicone substrate.

As will be described in more detail below, the mouthpiece 12 is operable to have a plurality of positions in order to define a plurality of airpaths through the breath sampling apparatus, or to prevent the flow of air into the apparatus. In particular, the mouthpiece is moveable between a first position shown in FIG. 17(i), a second position shown in FIG. 17(ii) and 25(i) and a third position shown in FIG. 17(iii) and 25(ii).

In the first position, the at least one mouthpiece outlet 18 is occluded, preventing airflow into a first sample chamber volume 15a of the sample chamber 14. In the example shown, the mouthpiece is located at a radial and axial position at which the mouthpiece outlets 18 are fully occluded by a wall 26 of the sample chamber, so as to be blocked, thus preventing airflow through said outlets. Air is also prevented from flowing into the sample tube when the mouthpiece is in the first position.

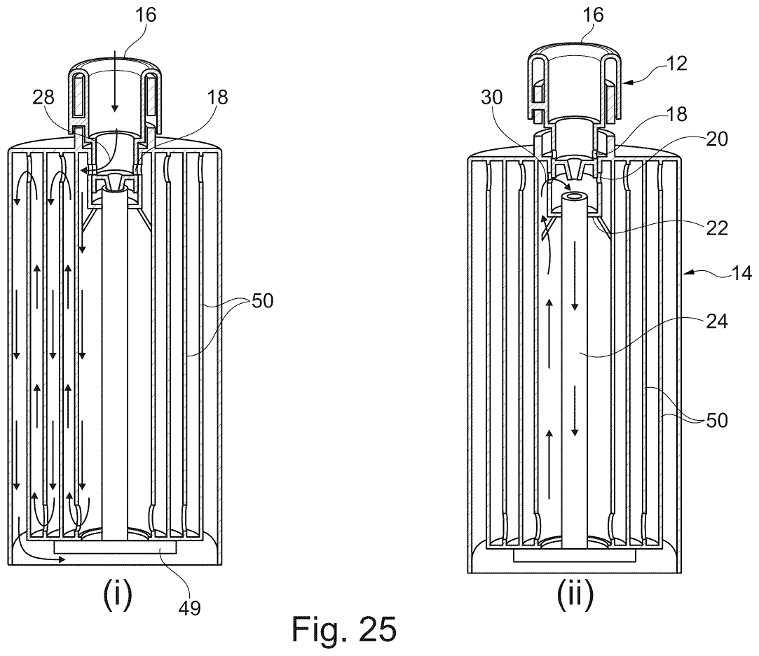

In the second position, the at least one mouthpiece outlet 18 is at least partly overlapping with the at least one chamber aperture 20, permitting air to flow along a first flow path (indicated by arrows 28 in FIG. 25(i)) from the mouthpiece inlet 16 through the at least one chamber aperture 20 into the sample chamber 14. In the example shown, the mouthpiece is located at a radial and axial position at which the mouthpiece outlets 18 are aligned with the chamber apertures 20 so as to permit airflow through the aligned apertures 18, 20 into the sample chamber 14. Air is prevented from flowing into the sample tube when the mouthpiece is in the second position (as described below).

In the third position, as in the first position, the at least one mouthpiece outlet 18 does not overlap with the at least one chamber aperture 20. However, the third position differs from the first position in that air is permitted to flow through a second flow path different to the first flow path (indicated by arrows 30 in FIG. 25(ii)) from the sample chamber through the at least one chamber aperture into a sample medium held in the receiving aperture and defining a second sample chamber volume 15b. Air is prevented from flowing into the sample chamber from the mouthpiece inlet in the third position.

When assembled together, the mouthpiece 12 and sample chamber 14 define a sample chamber assembly having a longitudinal axis 32. The mouthpiece 12 is rotatable in a first direction, indicated by arrow 34, between the first position and the second position. That is, the first and second mouthpiece positions have the same axial position with respect to the longitudinal axis of the sample chamber but differing radial positions.

The mouthpiece 12 is also displaceable in a second direction, indicated by arrow 36, between the second position and the third position. The second direction is different to the first direction, and in the example shown is parallel with the longitudinal axis 32. That is, the second and third mouthpiece positions have the same radial location with respect to the longitudinal axis of the sample chamber but differing axial positions. The third position lies beyond the second position, such that the mouthpiece must move through the second position to reach the third position.

Such an arrangement may be achieved in a manner similar to a bayonet fitting, as shown in FIGS. 18 and 19. In the specific and non-limiting example shown in these Figures, it can be seen that the wall 26 of a neck 38 of the sample chamber 14 includes a guide 40. The guide 40 includes a first guide portion 40a and a second guide portion 40b. The first guide portion 40a, in this example, substantially follows a plane that is perpendicular to the longitudinal axis 32, so as to define a circumferential path around the axis. A first end stop 41a determines the first position, and a second end stop 41b determines the second position. In a similar manner, the second guide portion 40b, in this example, is substantially parallel to the longitudinal axis 32, so as to define an axial path aligned with the axis 32. A third end stop 41c determines the third position.

As shown in FIG. 19, the mouthpiece includes one or more lugs 42 which are received in the guide, so that the mouthpiece location is restricted by the interaction between the lugs 42 and the guide portions 40a, 40b (see FIG. 18). Thus it can be seen that the guide 40 defines a stepped path which constrains movement of the mouthpiece 12 to the first and second directions 34, 36 by constraining the movement of the lugs 42.

The guide 40 is shown extending through the full thickness of the neck of the sample chamber, but it will be appreciated that this is not necessary and that the guide could be in the form of a channel extending only part way through the neck. It will further be appreciated that other guide shapes are possible, and that the lug and guide could be reversed if required, such that the lugs could be provided on the neck and the guide could be provided in the mouthpiece.

The example shown also includes a third guide portion 40c that permits the mouthpiece to be turned to a fourth position defined by a fourth stop 41 d, illustrated in FIG. 17(iv). From the fourth position the mouthpiece may be moved axially along a fourth guide portion 40d as illustrated in FIG. 17(v), and removed from the sample chamber, so permitting access to the interior of the sample chamber.

Turning back to FIGS. 17 and 19, the mouthpiece further comprises a sealing member 44 operable to seal against an inlet of a sample medium 24 held within the sample chamber 14. In the particular example shown, the sealing member is disposed on or comprised within an end stop 46, which prevents air from being exhaled directly into the sample tube when an individual exhales into the mouthpiece 12. Consequently, this inhibits condensate forming on the inlet of the sample medium 24, and prevents aerosol, and spital becoming entrained in the sample.

The end stop 46 has the shape of a truncated cone, so as to abut against an inlet of a sample medium held within the receiving aperture 22. An underside of the end stop 46 is configured to seal against the sample tube when the mouthpiece is in the first and second position, and also when transiting between those two positions. In the example shown, one or more resilient seals, such as small deformable ridges, are provided on the underside of the end stop 46 to ensure an airtight fit. Thus air flow into a sample medium held in the receiving aperture is prevented when the mouthpiece is in the first position, in the second position, or in between, either directly from the mouthpiece inlet 16 or indirectly via the sample chamber 14.

In the third position, the end stop is lifted axially up and out of the sample medium 24, permitting air to flow into the sample medium. At the same time the mouthpiece outlets 18 are moved out of alignment with the chamber apertures 20, so preventing air flow from the mouthpiece inlet into the sample chamber when the mouthpiece is in the third position. Air can thus only flow into the sample medium 24 from the sample chamber 14 (e.g. as shown in FIG. 8).

The sample chamber further includes one or more exhaust outlets 48, which may be covered by a filter 49 such as an FFP3 or N99 filter. The interior of the sample chamber is shaped to define a tortuous airflow path having an upstream end adjacent the chamber apertures 20 and a downstream end adjacent the exhaust outlets 48. In the particular example shown, the sample chamber comprises a plurality of nested concentric walls 50 defining a concertina-shaped airflow path. The chamber apertures 20 are adjacent the receiving aperture 22, such that both are adjacent the upstream end of the airflow path.

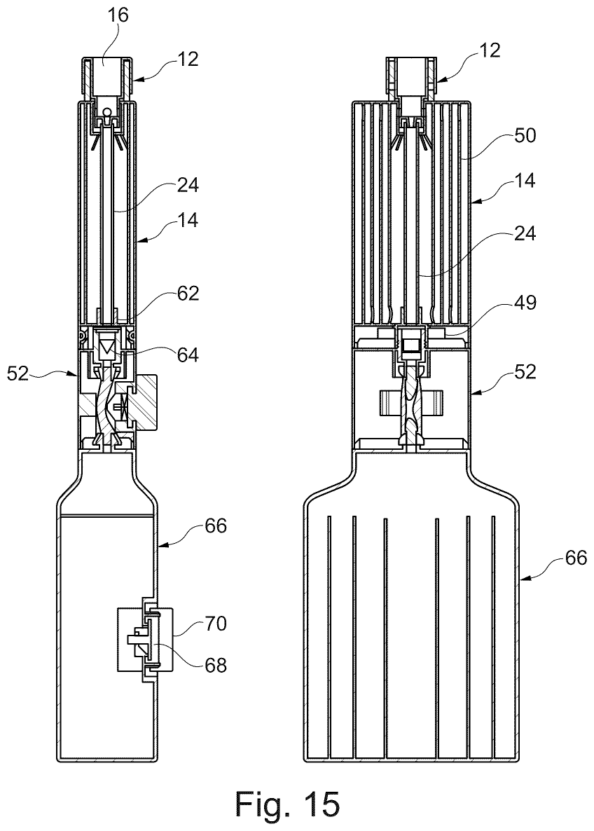

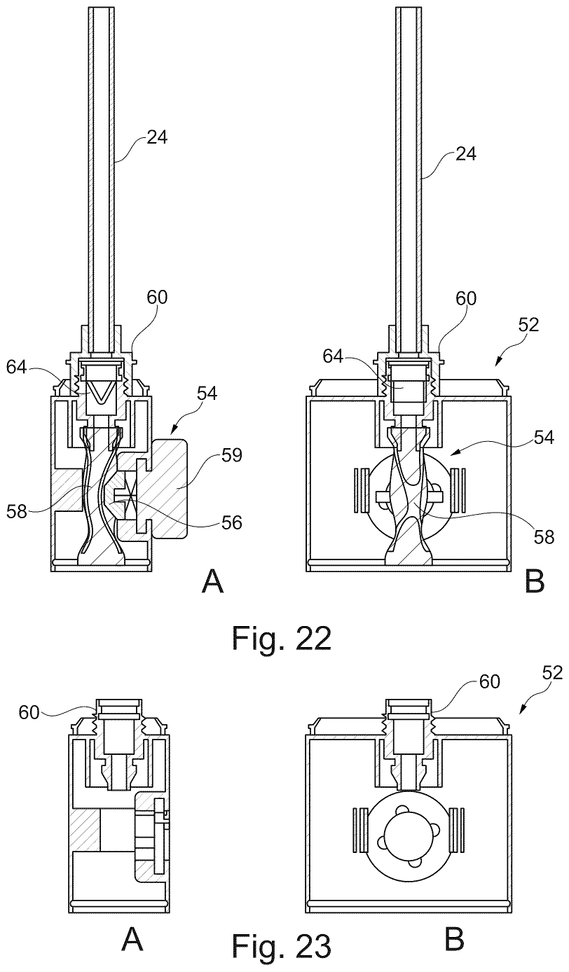

Referring now to FIGS. 15 and 22, the breath sample collection apparatus 10 further includes a first valve 54 fluidly downstream of a valve outlet 55 in the sample chamber 14. The first valve 54 has an open position in which air is permitted to flow through the first valve and a closed position in which air flow through the first valve is prevented. In the particular example shown, the first valve is a pinch valve which includes an actuator 56 and a tube 58 compressible by the actuator. A handle 59 is configured to interact with the actuator to permit a user to move the valve 54 between the open position and the closed position.

The first valve 54 is, in the example shown, located within a valve chamber 52 connectable to the sample chamber, for example via frictional engagement, such as a gas-tight snap fit. In the example shown, a deformable seal 53 is provided at an interface between the valve chamber 52 and the sample chamber 14 in order to ensure the frictional engagement between those two components is substantially sealed to ingress from external air.

The apparatus, and in particular the valve chamber 52, additionally includes an adapter 60 operable to couple to the sample medium 24. When the valve chamber 52 is connected to the sample chamber, the adaptor protrudes through an adapter aperture 62 (which, in the example shown, comprises the valve outlet 55) in the base of the sample chamber 14. The sample medium 24 may then be held within the sample chamber 14 by the adapter 62 at a downstream end and by the receiving aperture 22 at an upstream end. The adapter may be removable and replaceable, for example via a screw fitting, so that different adapters may be used depending on the type of sampling medium to be held within the sample chamber.

A non-return valve 64 is operable to permit air to flow through the adaptor 62 from the sample chamber 14 to the valve chamber 52 but to prevent air flow in the reverse direction. The non-return valve is depicted in FIG. 22 as a duck-bill valve having two resilient flaps, the free ends of which press together to form a gas-tight seal.

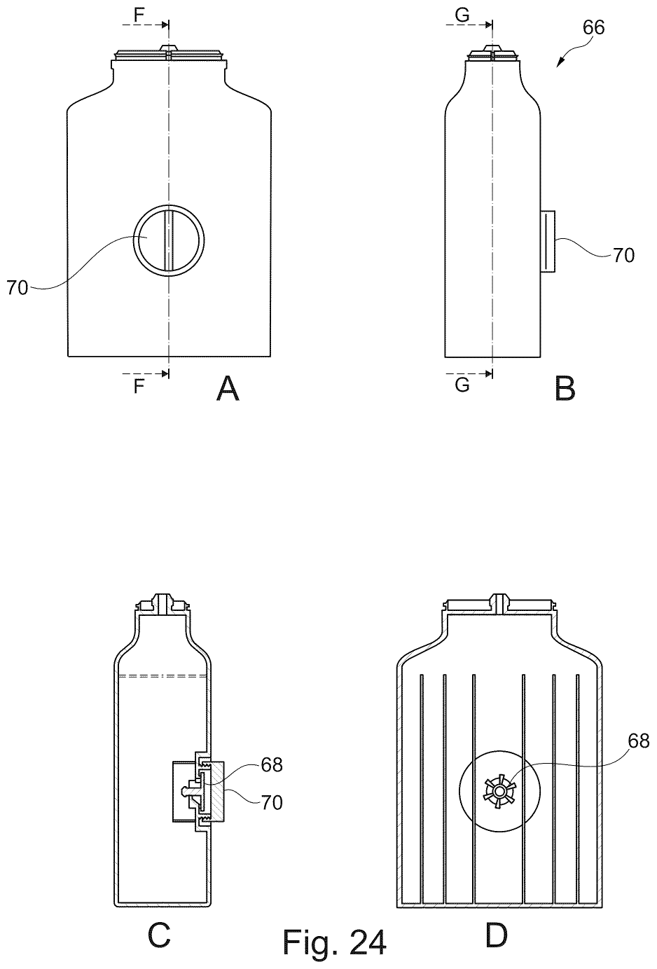

Referring now to FIGS. 13 and 24, the breath sample collection apparatus additionally includes a vacuum source. The vacuum source is operable, when fluidly connected to the sample chamber 14 with the mouthpiece 12 in the third position, to draw air within the sample chamber through a sample medium 24 held in the receiving aperture 22, i.e. to reverse the direction of airflow through the sample chamber 14. Examples of a suitable vacuum source include a pump or syringe fluidly connectable to the first valve 54 at a side of the valve remote from the sample chamber (i.e. so that the first valve 54 is between the vacuum source and the sample chamber). In the particular example shown, the vacuum source comprises a vacuum chamber 66 that is connectable to the valve chamber 52, for example via a frictional engagement including a deformable seal, so as to form a gas-tight snap fit. The first valve is then fluidly connected between the vacuum chamber 66 and the adapter 60, ensuring that the vacuum chamber 66 is only operable to draw air within the sample chamber through the sample medium 24 when the first valve is in the open position.

The vacuum chamber may be depressurised using a one way valve 68 which, in the example shown, is in the form of an umbrella valve. A removable dust cover 70 protects the umbrella valve when it is not in use.

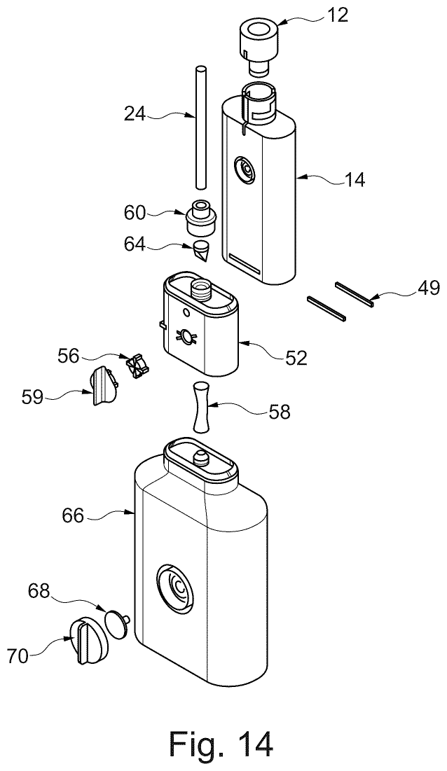

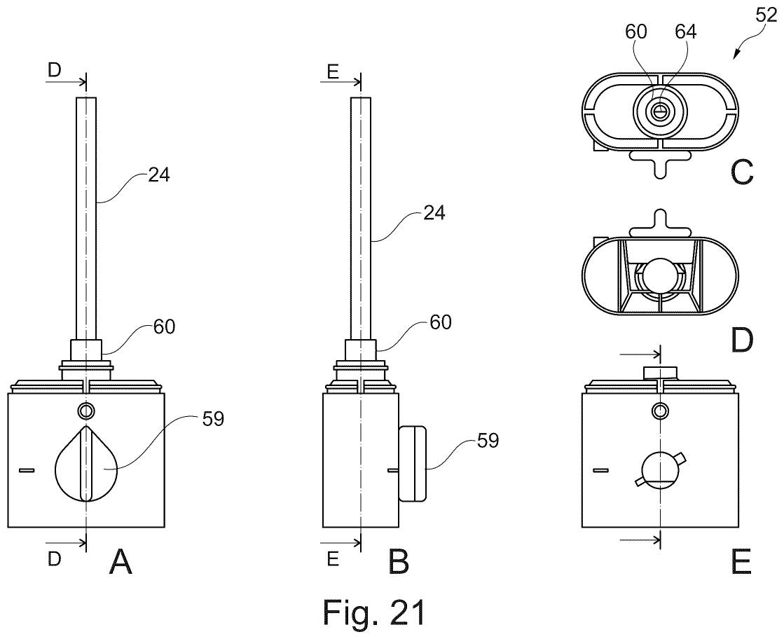

As explained above, the breath sampler illustrated in FIGS. 13-25 includes four snap together sub-assemblies, namely:
a valved mouthpiece 12
a sample chamber 14;
a valve mid-section 52; and
a vacuum chamber 66

As explained above, the single piece valved mouthpiece 12 does more than enable someone to breathe into the sample chamber. It also acts as a multiport valve that:
seals the sampling media before and after use;
isolates the sample chamber from the environment during sample transfer from the sampling chamber onto the sampling media;
protects the sampling media from condensate, particulate, and aerosol ingress; and,
when required it may be removed to convert the breath sampling apparatus into an environmental air sampler for either actively pumped sampling, or passive diffusive sampling, depending on the context.

As explained above, the sample chamber 14 contains:
a tortuous airflow path, in the form of a series of nested tubes connected so as to form a concertinaed breath sampling tube that operates in a similar manner to a Priestley-Haldane sampling tube;
an exhaust outlet from the airflow path protected by a N99 filter;
a two-stage bayonet fitting for the valved mouthpiece; and,
a gas-tight snap fit connector to the central valved section.

The filter within the sample chamber ensures that any infectious risk associated with the breath sample is contained and isolated within the sample chamber during transfer to a laboratory, where the sampling media may be recovered with appropriate biosecurity. Conversely, the sampling chamber also protects the sampling media from inadvertent environmental or handling contamination during use and transfer to the testing laboratory.

As explained above, the valve mid-section 52 contains:
a collection pinch valve including an actuator moveable to compress a length of silicone tubing;
an adaptor for fixing and sealing sample media in place within the sample chamber;
a non-return valve to prevent contamination of the sample media; and,
gas tight snap connectors to the sample chamber and vacuum chamber.

Twisting the collection valve handle causes the gate on the collection valve to grip or release the short length of silicone tubing. Releasing the grip will cause the vacuum in the vacuum chamber to draw air through the sample media. The adapter may be removable, permitting different adapters to be used with the apparatus, this in turn permits the apparatus to be used with different types of sampling media.

Finally, the vacuum chamber 66 is a reinforced chamber, for example a chamber having a honeycomb interior structure, that connects to the silicone tube in the valve mid-section. It is strong enough to hold at least a partial vacuum. A one-way flap valve enables a simple pump to be used to reduce the pressure in the chamber. Opening the collection valve causes the partial vacuum to draw the sampled volatiles into the sampling media. A dust cap protects the flap-valve.

The breath sampling apparatus may be made from any suitable clinically stable and sterilisable material, such as plastics or metal. The example depicted in the figures is made from polyethylene terephthalate glycol (PETG) and silicone which are moulded and printed into the four snap together sub-assemblies. Any conventional sample media may be fitted into an appropriately shaped adapter in the valve section, to be held within the sample chamber in use.

PETG is used because it:
is impact, and chemically resistant;
may be thermoformed and injection-moulded;
is FDA compliant, non-toxic, used widely for food and medical packaging as well as for laboratory sampling consumables;
has a very-low VOC out gassing profile;
is resistant to VOC adsorption and absorption artefacts;
it is a widely available commodity; and,
it is readily recyclable.

Silicone is used because it:
is FDA compliant, non-toxic, used widely for food and medical applications;
provides gas-tight seals and valve seats;
is a widely available commodity; that,
may be readily sterilised and reused.

Use of the breath sampling apparatus shown in FIGS. 13-25 is straightforward and easily repeatable. In order to use the apparatus an individual simply needs to blow gently into the valved mouthpiece until they have emptied their lungs. It is important that the individual does not breathe in through the breath sampler, as to do so may draw contaminated air into the apparatus and invalidate the breath sample. There is no need for the individual to exhale forcefully, or to breathe out for longer than is comfortable.

The apparatus will typically be supplied with the mouthpiece located in the first position (i.e. with all inlets sealed in order to protect the sample medium inside), and with a sampling medium already inside the sample chamber. To use the breath sampling apparatus the mouthpiece should be turned from the first position to the second position, to permit air to flow between the mouthpiece and the sample chamber. In the example shown in the Figures, the mouthpiece is moved into the second (sampling) position by twisting the valved mouthpiece anticlockwise until a stop is reached (second stop 41 b). This is about ⅛^{th} of a turn.

After asking the participant to take a breath, the sampling technician should immediately hand the breath sampler to the individual for them to blow into, until they have emptied their lungs. The individual's breath displaces the air in the sample chamber through the bio-filtered exhaust, as shown in FIG. 25(i). The individual should keep breathing out for as long as they can, until they have fully emptied their lungs. At this point the air around the sampling media and throughout the rest of the breath chamber is Phase III alveolar air from their expiratory reserve volume.

As soon as the participant has finished breathing out, the sample chamber should be sealed to prevent contamination. Typically, the individual should immediately return the breath sampler to the sampling technician, without breathing in through the apparatus. The sampling technician will then seal the sample chamber by moving the mouthpiece to the third position, for example by pulling the valved mouthpiece outwards (axially away from the sample chamber) until a stop is reached (third stop 41 c). In the example shown, this is about 1 cm, and the guide 40 is visible. Once the sample has been sealed, the collection valve may be actuated to the open position. In the example shown, this involves turning the valve handle a quarter turn clockwise. The vacuum in the vacuum chamber then draws the sampled breath into the sampler media. For a standard thermal desorption tube, approximately 50 cm3 of breath sample is drawn from the sample chamber through the chamber aperture and into the sample tube 24 (see FIG. 25(ii)). The sampling technician should wait a predetermined time to ensure that a sufficient volume of sample has been collected, so example 10-60 s, e.g. 30 s.

The collected sample may then be sent to a laboratory for processing, protected within the sealed sample collection chamber.

A breath sampling apparatus of the type described herein provides an expiratory reserve volume breath sampler, which may be single use, and which may be used anywhere to:
reproducibly sample a predefined last volume (e.g. the last 50 cm) of exhaled breath from an expiratory reserve volume breath sample, without requiring any forced breathing manoeuvres;
trap exhaled volatile organic compounds (VOC) and other respiratory gasses onto an adsorbent trapping device;
provide flexibility in the choice of sampling media in an adsorbent trap, with 3.5″ industry standard thermal desorption tubes, needle-trap, SPME and silicone substrate options all compatible;
obtain a breath sample in less than 30 s from start to finish;
be intrinsically safe with an N99 viral filter to trap potential exhaled pathogens;
be intuitive to use, requiring less than 5 min training;
isolate the sampling media and protect the breath sample from inadvertent contamination due to handling and environmental exposures;
have a minimised environmental impact with recyclable and reusable materials used throughout;
protect the sample from entrained bioaerosol and condensate; and above all,
provide high quality breath data at a competitive price.

A breath sampling apparatus and method have been described above by way of example. It will be appreciated however that modifications may be made to the apparatus and methods described herein where appropriate. For example, it will be appreciated that the shape, number and location of the mouthpiece inlet, mouthpiece outlets, chamber inlet regions/apertures and receiving region/aperture may all be varied according to design preference, without altering their function. Features shown in the drawings may be omitted if not essential, or may be replaced by equivalent features. The invention is defined in the following claims.

1. A handheld device for collecting a fluid and storing a sample of or from the fluid, the device comprising:
a first sample chamber volume having a first flow path for receiving the fluid;
first selective closure means for selectively closing the first flow path so that fluid may not flow into or out of the first sample chamber volume via the first flow path;
a second sample chamber volume in selective fluid communication with the first sample chamber volume;
second selective closure means for selectively fluidly isolating the second sample chamber volume from the first sample chamber volume; and
fluid moving means for moving fluid from the first sample chamber volume to the second sample chamber volume when the second selective closure means permits fluid flow therebetween;
wherein the device is moveable between:
a first configuration in which fluid may flow into in the first sample chamber volume via the first fluid path and be collected therein whilst the second selective closure means fluidly isolates the second sample chamber volume from the first sample chamber volume; and
a second configuration in which the first fluid path is closed by the first selective closure means, the second selective closure means permits fluid flow between the first sample chamber volume and the second sample chamber volume, and the fluid moving means causes the collected fluid to move into the second sample chamber volume so that a sample of or from the fluid may be stored in the second sample chamber volume.

2. The device of claim 1, wherein the device is moveable between a pre-use configuration and the first configuration and second configuration, wherein in the pre-use configuration, the first fluid path is closed by the first selective closure means and fluid flow between the first sample chamber volume and the second sample chamber volume is prevented by the second selective closure means.

3. The device of claim 1, wherein the first selective closure means is manually moveable between a closed position in which the first fluid path is closed and an open position in which the first fluid path is open.

4. The device of claim 1, wherein the second selective closure means is manually moveable between a closed position in which the second sample chamber volume is fluidly isolated from the first sample chamber volume, and an open position in which the second sample chamber volume is in fluid communication with the first sample chamber volume.

5. The device of claim 4, wherein the first selective closure means is manually moveable between a closed position in which the first fluid path is closed and an open position in which the first fluid path is open, and the device comprises an inlet collar wherein at least part of the first selective closure means and/or the second selective closure means form part of the inlet collar.

6. The device of claim 5, wherein the inlet collar is rotatably moveable so as to move the first selective closure means between the closed position and the open, and/or wherein the inlet collar is axially moveable so as to move the second selective closure means between the closed position and the open position.

7. (canceled)

8. The device of claim 7, wherein the inlet collar is rotatably moveable so as to move the first selective closure means between the closed position and the open, and wherein the inlet collar comprises a mouthpiece and the device is a device for collecting breath and storing a sample of or from the breath.

9. The device of claim 8, wherein the mouthpiece has a mouthpiece inlet and at least one mouthpiece outlet, and the first sample chamber volume has at least one chamber aperture;
wherein the mouthpiece is moveable between:
a first position in which the at least one mouthpiece outlet is occluded, thereby closing the first flow path;
a second position in which the at least one mouthpiece outlet is at least partly overlapping with the at least one chamber aperture, permitting air to flow through the first flow path from the mouthpiece inlet through the at least one chamber aperture into the first sample chamber volume; and
a third position in which the at least one mouthpiece outlet is not overlapping with the at least one chamber aperture, permitting air to flow through a second flow path from the first sample chamber volume through the at least one chamber aperture into the second sample chamber volume.

10. The device of claim 9, wherein the third position lies beyond the second position, such that the mouthpiece must move through the second position to reach the third position.

11. The device of claim 9, wherein the mouthpiece is rotatable in a first direction between the first position and the second position, and optionally wherein the mouthpiece is displaceable in a second direction between the second position and the third position, the second direction being different to the first direction.

12. (canceled)

13. The device of claim 9, wherein the mouthpiece further comprises a sealing member operable to seal against an inlet of a sample medium.

14. The device of claim 9, wherein air flow into a sample medium held in the receiving aperture is prevented when the mouthpiece is in the first position or in the second position.

15. The device of claim 9, wherein air flow from the mouthpiece inlet into the first sample chamber volume is prevented when the mouthpiece is in the first position or in the third position.

16. (canceled)

17. The device of claim 9, further comprising a first valve fluidly connected downstream of a valve outlet of the second sample chamber volume, the first valve having an open position in which air is permitted to flow through the first valve and a closed position in which air flow through the first valve is prevented.

18-19. (canceled)

20. The device of claim 9, wherein the fluid moving means comprises a vacuum source operable, when fluidly connected to the second sample chamber volume with the mouthpiece in the third position, to draw air within the first sample chamber volume through a sample medium held in the receiving aperture.

21. The device of claim 1, wherein the fluid moving means comprises a vacuum source, such that when the vacuum source is in fluid communication with the second sample chamber volume a pressure difference causes the collected fluid to move into the second sample chamber volume.

22. The device of claim 21, wherein the vacuum source comprises a vacuum chamber containing a vacuum or a gas having lower than atmospheric pressure.

23-25. (canceled)

26. The device of claim 1, wherein the second sample chamber volume comprises a sample medium.

27. The device of claim 1, wherein the first sample chamber volume further comprises one or more exhaust outlets, said exhaust outlet optionally or preferably comprising a filter.

28. The device of claim 27, wherein the first sample chamber volume comprises a tortuous airflow path having an upstream end adjacent the first flow path and a downstream end adjacent the at least one exhaust outlet.

29. (canceled)
